(19) United States
(12) Patent Application Publication
(10) Pub. No.: US 2020/0179156 A1
(43) Pub. Date: Jun. 11, 2020

(54) URINARY INCONTINENCE DEVICE

(71) Applicant: Torfrieda Alma Vlet, Union, NJ (US)

(72) Inventor: Torfrieda Alma Vlet, Union, NJ (US)

(21) Appl. No.: 16/793,657

(22) Filed: Feb. 18, 2020

Related U.S. Application Data
(63) Continuation of application No. 15/652,480, filed on Jul. 18, 2017.
(60) Provisional application No. 62/363,628, filed on Jul. 18, 2016.

(51) Int. Cl. A61F 5/453; A61F 2/00

(52) U.S. Cl. CPC A61F 5/453; A61F 2/0009

(57) ABSTRACT

A urinary incontinence device is provided. The incontinence device may generally be comprised of a pouch having an absorbent material therein. An outer material, that is impervious to liquid, retains the absorbent material as well as any liquid excreted by the patient. The device may be further used in conjunction with a waist strap or harness to further support the device.

### CLAIM OF PRIORITY

This application is a Continuation-in-Part Patent Application of U.S. Non-Provisional patent application Ser. No. 15/652,480 filed on Jul. 18, 2017, which claims priority to U.S. Provisional Patent Application Ser. No. 62/363,628 filed on Jul. 18, 2016, the contents of which are herein fully incorporated by reference in their entirety.

### FIELD OF THE EMBODIMENTS

The field of the embodiments of the present invention is related to devices for urinary assistance and/or incontinence. In particular, the embodiments provide for a superior to traditional catheters.

### BACKGROUND OF THE EMBODIMENTS

A wide variety of incontinence products have been developed for the incontinent adult. Incontinence pads, diapers, and other garments have been used for many years to alleviate the practical and hygienic problems encountered by individuals suffering from incontinence issues. Normally, these diapers have one or more inner layers of absorbent material contained in an outer layer of waterproof fabric, so that any liquid (urine) excreted by the patient is retained by the diaper and prevented from leaking outside the areas of contact with the patient's body. However, as many of such devices allow for contact between a patient's skin and wet material, skin irritation, rashes, bed sores, ulcers, and the like are common afflictions of the wearer. In some examples, bed sores, if not taken care of, turn into a hole that eats flesh away due to the prolonged exposure to urine.

Further, the absorbent material within the diaper is typically incapable of adequately rapidly absorbing a high flow rate of voided urine. Consequently, the urine will often overflow the diaper, resulting in the wetting of the wearer's clothes. Additionally, these diapers are best suited for women where the diaper bag opening can be located proximate the urethra to properly receive urine. These diapers are a problem for males wherein the position of the urethra outlet may not be directed into the ideal position at the time of voiding.

In other instances, catheters may be employed to drain urine from the bladder from patients. However, catheters can be extremely uncomfortable and cause numerous painful side effects. For example, men sometimes have bladder spasms while the catheter is in their penis. These spasms may be caused by the catheter bothering the bladder. In other instances, there may be blood in the urine. It is normal for the urine draining from your catheter to have some small blood clots. In addition, infections of the urinary tract are very common. These are only side effects caused by the catheter, whereas one must also keep collection bag clean and prevent tugging on the catheter save for causing potentially much more serious issues. It should be appreciated that if the user is not kept clean and dry, the user can face additional health illnesses or diseases.

Thus, there is a need for a comfortable and effective urine retainment device that provides confidence and dignity to the wearer. Such a device should rapidly absorb urine and minimize contact of the skin with the urine to prevent skin irritation, rashes, bed sores, ulcers, and holes. The present invention and its embodiments meet and exceeds these objectives.

### Review of Related Technology:

U.S. Pat. No. 6,569,135 pertains to a urine absorbent pouch for male incontinence which completely surrounds the patient's penis, thereby minimizing the patient's exposure to wetness. The pouch is constructed from a piece of material which has a hole portion and a continuous portion, the hole portion containing a hole which is adapted to receive a patient's penis. A plurality of sealing means are located along the edges of the hole portion so when the sealing means are sealed onto the continuous portion, there is minimal contact between the sealing means and the patient. Additionally, a urine absorbent pouch for male incontinence can be created from a commercially available baby diaper by cutting a hole in the diaper and attaching additional sealing means to the portion of the diaper with the hole.

U.S. Pat. No. 5,094,853 pertains to a novel arthropodicidally-active composition-of-matter. Such comprises an aqueous lower alkanol solvent, a toxicant contained within the solvent, and an effective amount of an emulsifier, also contained within the solvent, for forming a foam matrix of predetermined stability. Also disclosed are methods for producing such a composition-of-matter.

U.S. Pat. No. 4,559,051 pertains to a disposable diaper having improved liquid receiving and retaining capabilities. The diaper includes a waterproof barrier formed as a flattened bag and having a single opening located adjacent the perineal area of the body of the wearer. Two layers of filler material are positioned within the barrier bag. The upper layer adjacent the opening is a wicking material, while the lower layer is a superabsorbent material. In a male version of the diaper, a pouch is formed over the opening into which the wearer's penis is inserted to direct voided urine through the bag opening.

Various devices are known in the art. However, their structure and usage are substantially different from the present disclosure. The other inventions also fail to solve all the problems taught by the present disclosure. The embodiments of this invention are presented in the drawings below and will be described in more detail herein.

### SUMMARY OF THE EMBODIMENTS

Generally, the embodiments of the present invention provide for a male incontinence device that may be worn by a user as needed, such as, before, during, or after surgery or by those generally having incontinence issues. Some embodiments of the instant invention may be worn by infants to keep the infant dry.

A first embodiment of the instant invention describes a urinary incontinence device. The device includes a pouch having at least one inner layer and an outer layer defining an opening. An adhesive may be disposed on an outer surface of the pouch. The opening is configured to receive a human penis and is size adjustable. One of the at least one inner layer is comprised of an absorbent material. The absorbent material contains sodium polyacrylate. The outer layer is made of a liquid impervious material.

The device also includes at least one securement mechanism coupled to the pouch and at least one retainment mechanism configured to be coupled to the pouch. The securement mechanism comprises a first end and a second end with each of the first end and the second end coupled to the pouch thereby forming a loop. In some examples, there are two securement mechanisms such that the at least one retainment mechanism is coupled to each of the two securement mechanisms. The device may further include flaps disposed in proximity to the opening and being configured to adjust the size of the opening.

A second embodiment of the instant invention describes a male urinary incontinence device. The device is configured to hold up to 1 L of fluid. The device includes a pouch having an inner surface and an outer surface defining an opening with the opening being configured to receive a human penis. An absorbent material is disposed between the inner surface and the outer surface. The outer surface comprises an outer layer and an inner layer. The outer layer comprises a liquid impervious material. The inner layer comprises a cloth layer.

The device also includes two securement mechanisms coupled to the pouch such that each of the two securement mechanisms comprises a first end and a second end with each of the first end and the second end coupled to the pouch thereby forming a loop. The device further includes a length of material having a first end and a second end with the first end being configured to be coupled to one of the two securement mechanisms and the second end being configured to be coupled to another of the two securement mechanisms. The length of material has a fastening mechanism disposed on each of the first end and the second end of the length of material.

A third embodiment of the instant invention describes a male urinary incontinence device. The device includes a pouch having at least one inner layer and an outer layer defining an opening with the opening being configured to receive a human penis. A base of the pouch is wider than the opening of the pouch. The opening comprises a slat configuration or a door configuration affixed to a concave portion such that the human penis rests on the concave portion once the human penis is received through the opening.

The at least one inner layer is comprised of sodium polyacrylate and is removable. The outer layer is made of a petroleum-based material and is about 3 inches thick. The device also includes at least one flap disposed in proximity to the opening and being configured to adjust the size of the opening. A positioning mechanism is disposed on one end of the at least one flap to secure a position of the at least one flap.

The device also includes two securement mechanisms coupled to the pouch, such that each of the two securement mechanisms comprises a first end and a second end with each of the first end and the second end being coupled to the pouch to form a loop. The device further includes an adhesive material disposed on an outer layer of the pouch. The adhesive material is disposed on a back surface of the pouch.

The device further includes a length of material having a first end and a second end with the first end being coupled to one of the two securement mechanisms and the second end being coupled to another of the two securement mechanisms. The length of material has a fastening mechanism disposed on each of the first end and the second end of the length of material.

The device also includes a receiving pouch affixed to the opening on an outer surface of a front side of the device. The receiving pouch is configured to receive and house the human penis once received through the opening. The receiving pouch comprises one or more urine-absorbent materials.

In general, the present invention succeeds in conferring the following, and others not mentioned, benefits and objectives.

It is an object of the present invention to provide a device that removes the need for a traditional catheter.

It is an object of the present invention to provide a device that prevents spillage or urine and/or other bodily fluids.

It is an object of the present invention to provide a device that is easy to put on and take off.

It is an object of the present invention to provide a device that is disposable.

It is an object of the present invention to provide a device that is a one size fits all device.

It is an object of the present invention to provide a device that is lightweight and comfortable.

It is an object of the present invention to provide a device that allows a user to be mobile while using the device.

It is an object of the present invention to provide a device that can be secured or otherwise supported to provide additional comfort to the user.

It is an object of the present invention to provide a comfortable and effective urine retainment device that provides confidence and dignity to the wearer.

It is an object of the present invention to provide a device that rapidly absorbs urine and minimizes contact of the skin with the urine to prevent skin irritation, rashes, bed sores, ulcers, and holes.

### BRIEF DESCRIPTION OF THE DRAWINGS

FIG. 1 depicts a front perspective view of a first embodiment of the present invention.

FIG. 2 depicts a front perspective view of a first embodiment with an attached support of the present invention.

FIG. 3 depicts a cutaway side view of a first embodiment of the present invention.

FIG. 4 depicts a front perspective view of a first embodiment of the present invention in use.

FIG. 5 depicts a perspective view of a urinary incontinence device of a second embodiment of the present invention.

FIG. 6 depicts another perspective view of a urinary incontinence device of a second embodiment of the present invention.

FIG. 7 depicts another perspective view of a urinary incontinence device of a second embodiment of the present invention.

FIG. 8 depicts a perspective view of a retainment mechanism of a urinary incontinence device of a second embodiment of the present invention.

FIG. 9 depicts a front perspective view of a urinary incontinence device of a third embodiment of the present invention.

FIG. 10 depicts a rear perspective view of a urinary incontinence device of a third embodiment of the present invention.

FIG. 11 depicts a perspective view of a receiving pouch of the present invention.

FIG. 12 depicts a perspective view of a system comprising the receiving pouch of FIG. 11 and the urinary incontinence device of the third embodiment of FIG. 9 and FIG. 10 of the present invention.

FIG. 13 depicts another perspective view of a system comprising the receiving pouch of FIG. 11 and the urinary incontinence device of the third embodiment of FIG. 9 and FIG. 10 of the present invention.

### DETAILED DESCRIPTION OF THE EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification that various modifications and variations can be made thereto.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

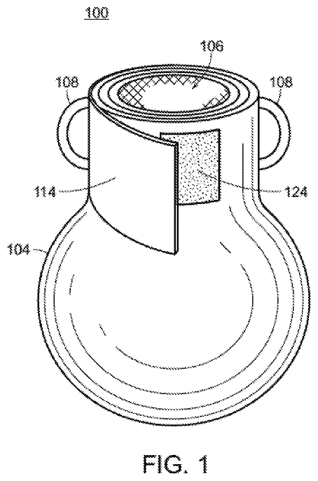

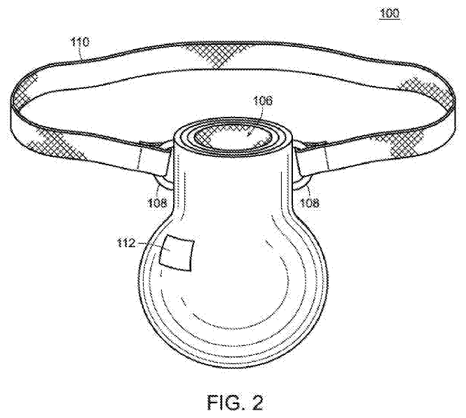

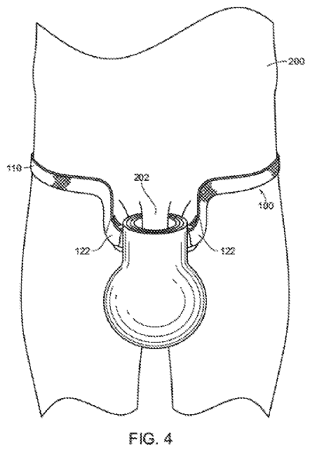

FIG. 1-FIG. 4 depict numerous views of a first embodiment of a device 100 of the present invention. The device 100 of the instant figures is worn externally by the wearer, and thus, provides enhanced benefits not afforded by a catheter. This first embodiment is designed to be worn by individuals who are mobile. Generally, the device 100 has an opening 106, securement mechanisms 108, an attachment mechanism 124, a flap 114, an outer surface 104, a retainment mechanism 110 (of FIG. 2 and FIG. 4), and an adhesive area 112 (of FIG. 2).

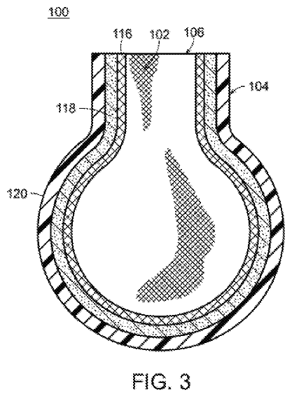

The device 100 is shaped to form a pouch having an inner surface 102 (as depicted in FIG. 3) and an outer surface 104 (of FIG. 1 and FIG. 3). Between the inner surface 102 (of FIG. 3) and the outer surface 104 (of FIG. 1 and FIG. 3) are a number of layers of various materials. The number of layers of the various materials may vary and may include about one layer to about ten layers, and more preferably may include about two layers to about four layers. Generally, the inner layer(s) comprise an absorbent material and the outer layer comprises a liquid impervious material. The pouch may have an overall length of about three inches to about twelve inches. The pouch also has a circumference of about eight inches. It should be appreciated that the dimensions provided herein are non-limiting and non-exhaustive and other dimensions are contemplated.

The pouch has an opening 106, which is sized to be configured to receive a human penis 202 (as depicted in FIG. 4) and to collect urine from a patient 200 (as depicted in FIG. 4), whether the patient 200 (as depicted in FIG. 4) is sick, bedridden, or incontinent. The opening 106 may be adjustable by way of, at least one, and in some embodiments, two flaps 114 (as depicted in FIG. 1). In other embodiments, there is an elastic material that surrounds the opening 106 that is configured to accommodate up to ten inches in diameter allowing for easy application and removal of the device 100.

The flap(s) 114 (of FIG. 1) have a positioning material located on a surface thereof and there is, in some embodiments, a complementary positioning material disposed on the outer surface 104 (of FIG. 1 and FIG. 3) of the device 100. By tightening or pulling on the flaps 114 (of FIG. 1) and coupling the flaps 114 (of FIG. 1) to the pouch, the size of the opening can effectively be changed. This allows the opening to accommodate the human penis 202 (as depicted in FIG. 4) of varying sizes while remaining securely attached to the patient 200 (as depicted in FIG. 4).

It should be appreciated that a base or lower area of the pouch is wider than a neck area of the pouch, with the neck area being in proximity to the opening 106. The neck area of the pouch may be comprised of differing or additional materials configured to be comforting to the patient 200 (as depicted in FIG. 4) using the device 100. It is intended that the neck area will have a length configured to accommodate a shaft of the human penis 202 (of FIG. 4) inserted therein. This allows for patient 200 (of FIG. 4) to minimize any contact with the “collection” area of the pouch as it begins to accumulate liquid waste therein.

Securement mechanisms 108 (of FIG. 1 and FIG. 2) are coupled to an outer surface 104 (of FIG. 1 and FIG. 3) of the device 100. Preferably these securement mechanisms 108 (of FIG.

1 and FIG. 2) are coupled to the neck area of the pouch, however, the securement mechanisms 108 (of FIG. 1 and FIG. 2) may be coupled anywhere on the outer surface 104 (of FIG. 1 and FIG. 3) of the device 100. In at least one embodiment, the securement mechanisms 108 (of FIG. 1 and FIG. 2) comprise a first end and a second end, with each of the first end and the second end being coupled to the device 100 thereby forming a loop. In yet another embodiment, the securement mechanisms 108 (of FIG. 1 and FIG. 2) are elastic in nature. In a preferred embodiment, there are two securement mechanisms 108 (of FIG. 1 and FIG. 2) with one securement mechanism 108 (of FIG. 1 and FIG. 2) being coupled to the device 100 on an opposing side of the device 100. In another embodiment there is only one securement mechanism 108 (of FIG. 1 and FIG. 2) disposed on the device 100.

A retainment mechanism 110 (of FIG. 2 and FIG. 4) is utilized to further secure the pouch to the patient 200 (of FIG. 4). Here, as depicted in FIG. 2 and FIG. 4, the retainment mechanism 110 takes the form of a belt. In some examples, the belt may have a thin material on a front side and/or a back side of the belt. In other examples, the retainment mechanism 110 may be separable and may attach to the device 100 (e.g., via Velcro or another fixation means) each time the device 100 is used. In some examples, the retainment mechanism 110 may be affixed to the device 100 such that it is not separable from the device 100. In some examples, the retainment mechanism 110 is optional.

The retainment mechanism 110 is further size adjustable to accommodate a waist area of the patient 200 (of FIG. 4). As various patients can fluctuate from one to another in size, a fastening mechanism, such as a hook and loop fastener, may be utilized to provide differing sizes of the retainment mechanism 110. Further, each of a first end and a second end of the retainment mechanism 110 is coupled to one of the two securement mechanisms 108 (of FIG. 1 and FIG. 2). This forms a complete device 100 consisting primarily of the pouch and the retainment mechanism 110.

An adhesive area or patch 112 (of FIG. 2) may further be present on an outer surface 104 (of FIG. 1 and FIG. 3) of the device 100. In some embodiments, a non-stick covering may be present on the adhesive area 112 (of FIG. 2) until it is desired to utilize the adhesive area 112 (of FIG. 2). In some embodiments, the adhesive area 112 (of FIG. 2) is on a front of the device 100, whereas in other embodiments it is on the side or rear of the device 100. In other embodiments, the adhesive area 112 (of FIG. 2) is present at more than one location on the device 100.

Generally, when desired, the adhesive area 112 (of FIG. 2) can be exposed, allowing the adhesive to be affixed to an article of clothing of the user. In other embodiments the adhesive area 112 (of FIG. 2) may be coupled directly to a skin surface of the patient 200 (of FIG. 4). This provides an additional point of contact to alleviate any strain or stress placed on the device 100 or the user as the device 100 becomes saturated. The adhesive area 112 (of FIG. 2) may be utilized in conjunction with the retainment mechanism 110 (of FIG. 2 and FIG. 4) or may be utilized solely on its own.

Referring now specifically to FIG. 3, a sectional side view of the device 100, namely the pouch, is shown. Here, the layers of the pouch are visible, as are the interrelationships between those layers. The pouch generally has an inner surface 102, an absorbent layer 118, a cloth layer 116, an outer surface 104, and a petroleum based (plastic) layer 120.

In use, the outer surface 104 comprises a liquid impervious material. The outer surface 104 abuts the plastic layer 120 and may be integrated with the plastic layer 120. In some embodiments, the outer surface 104 is simply an outer surface of the plastic layer 120. The plastic layer 120 is preferably about 0.5 inches to about 3 inches in thickness. In yet other embodiments, this outer surface 104 is a distinct and separate layer.

The inner surface 102 comprises a liquid absorbent material and abuts a cloth layer 116. The inner surface 102 may be integrate with the cloth layer 116. In some embodiments, the inner surface 102 is simply an inner surface of the cloth layer 116. In yet other embodiments, this is a distinct and separate layer.

Between the plastic layer 120 and the cloth layer 116 is an absorbent layer 118. The absorbent layer 118 is preferably comprised of sodium polyacrylate, but may be comprised of a blend of materials configured to readily absorb moisture. In at least one embodiment, it is desired that the absorbent layer 118 is capable of absorbing up to 2 L of fluid and more preferably up to 1 L of fluid.

In other embodiments, the absorbent layer 118 and the cloth layer 116 are separable from the plastic layer 120. In some instances, an additional layer, between the absorbent layer 118 and the plastic layer 120 exists such that this removal is facilitated without losing the liquid collected therein. In yet other embodiments, additional absorbent material may be disposed on the cavity formed in the pouch 107. However, such material may be provided for comfort purposes as generally it is desired to pull moisture away from the skin surface.

Specifically, FIG. 4 illustrates a preferred implementation of a first embodiment of the present invention. Here, the device 100 is shown coupled to the patient 200. In practice, the human penis 202 of the patient 200 is inserted in the opening 106 (see FIG. 1). The retainment mechanism 110 is disposed around a waist of the patient 200 and the fastening mechanism(s) 122 is manipulated such that the device 100 is held in place. The device 100 can then be held in place until it has become saturated and needs to be changed or until it is otherwise desirable to remove the device 100.

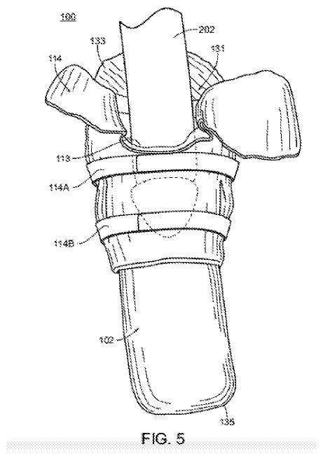

FIG. 5-FIG. 8 depict various views of a urinary incontinence device 100 of a second embodiment of the present invention. It should be appreciated that the second embodiment is substantially similar to the first embodiment described in FIG. 1-FIG. 4 and contains substantially similar components. However, the second embodiment may be used for bedridden males and infant males. Specifically, the second embodiment may be useful for individuals who are not mobile.

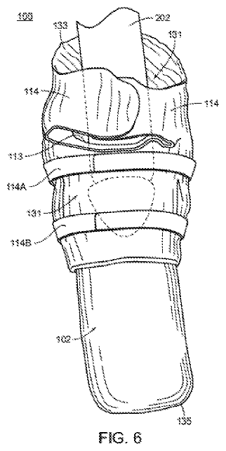

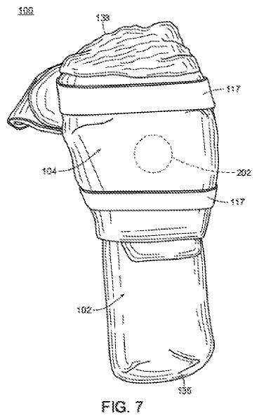

As shown in FIG. 5, FIG. 6, and FIG. 7, the second embodiment of the device 100 may be a diaper style device whereby a first end 133 contacts a front side of the patient 200 (of FIG.

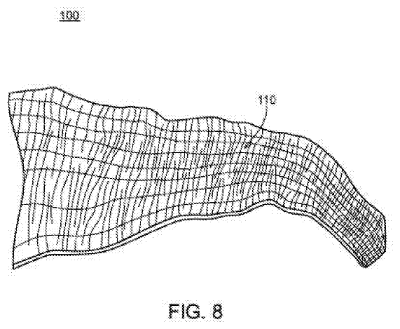

4) and a second end 135 contacts a back side of the patient 200 (of FIG. 4). The device 100 may include an opening 113 comprising a non-slip or rubber band-type material configured to receive the human penis 202 (of FIG. 4) therethrough. The retainment mechanism 110 (as depicted in at least FIG. 8) may be secured via a securement mechanism to an outside of the second end 135 to affix the device 100 on the patient 200 (of FIG. 4). In other examples, the retainment mechanism 110 is a belt having a large width to ensure comfort for the patient 200 (of FIG. 4) and may comprise a flexible or stretchable material.

In other examples, the retainment mechanism 110 (of FIG. 8) may be separable and may attach to the device 100 (e.g., via Velcro or another fixation means) each time the device 100 is used. In some examples, the retainment mechanism 110 (of FIG. 8) may be affixed to the device 100 such that it is not separable from the patient 200 (of FIG. 4). The retainment mechanism 110 (of FIG. 8) is also size adjustable to accommodate a waist area of the patient 200 (of FIG. 4).

Moreover, one or more straps 117 (of FIG. 7) may be located on the outer surface 104 (as depicted in FIG. 1 and FIG. 3) of the device 100. The one or more straps 117 (of FIG. 7) may be configured to affix the pouch in a closed position once the human penis 202 (of FIG. 4) is received therein.

Further, between an inner surface 102 (of FIG. 5, FIG. 6, and FIG. 7) and the outer surface 104 (of FIG. 7) of the device 100, one or more layers 131 (of FIG. 5 and FIG. 6) of various materials may be present. The one or more layers 131 (of FIG. 5 and FIG. 6) may comprise one or more hypoallergenic materials. In other examples, the one or more layers 131 (of FIG. 5 and FIG. 6) may comprise one or more non-woven hypoallergenic materials. In some examples, the one or more layers 131 (of FIG. 5 and FIG. 6) may be chemically treated to be odor-secure. The number of the one or more layers 131 (of FIG. 5 and FIG. 6) may vary and may preferably include a range of layers from about two layers to about four layers. Generally, the inner layer(s) comprise an absorbent material and the outer layer comprises a liquid impervious material.

Moreover, the device 100 may include flaps 114A, 114B (as shown in FIG. 5-FIG. 6). In some examples, the flaps 114A, 114B may comprise an elastic or a Velcro material. By tightening or pulling on the flaps 114A, 114B and coupling the flaps 114A, 114B to the device 100, the size of the opening 106 (of FIG. 1) can effectively be changed. This allows the opening to accommodate the human penis 202 (of FIG. 4) of varying sizes, while remaining securely attached to the patient 200 (of FIG. 4).

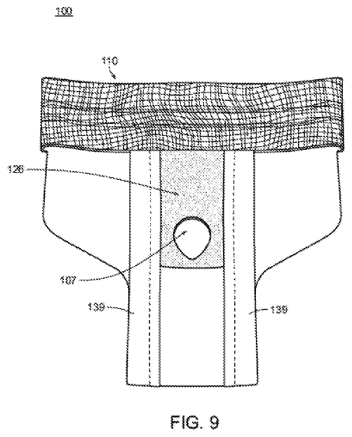

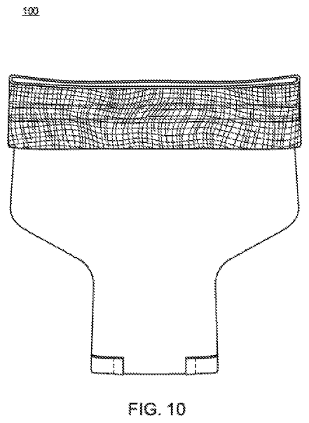

FIG. 9-FIG. 10 depict various views of a urinary incontinence device (e.g., the device 100) of a third embodiment of the present invention. It should be appreciated that the third embodiment is substantially similar to the first embodiment and the second embodiment described infra.

The third embodiment of FIG. 9-FIG. 10 may be used for men who are incontinent, bedridden, or are recovering from a surgery, as well as for infant care. In some examples, the device 100 replaces a need for a catheter for an adult recovering from surgery, as the device 100 may collect urine and keep the user dry. When used for infant care, the opening 107 may be covered by a material. Moreover, when used for infant care, the device 100 may comprise soft materials, such as soft plastic materials and soft Velcro materials, among other materials not explicitly listed herein. The device 100 helps contain urine and/or fecal matter and ensures that the urine and/or fecal matter do not mix. This helps keep the infant or adult male dry for approximately twelve hours.

As depicted in FIG. 9 and FIG. 10, the retainment mechanism 110 is configured as a belt, which may have a thin material on a front side and/or a back side of the belt. In other examples, the retainment mechanism 110 may be separable and may attach to the device 100 (e.g., via Velcro or another fixation means) each time the device 100 is used. In some examples, the retainment mechanism 110 may be affixed to the device 100 such that it is not separable from the device 100. In examples, the retainment mechanism 110 may be useful for mobile individuals and may prevent rolling of the device 100 on larger individuals.

In some examples and as depicted in FIG. 9, the one or more layers 131 (of FIG. 5) may comprise a sturdy and absorbent material centered in the device (e.g., a material 126), which is surrounded one each side by a lightweight material 139. It should be appreciated that the absorbent material, such as the material 126, may be removable from the device 100 once saturated with urine.

In some examples, and as depicted in FIG. 9, the device 100 may have an opening 107, which is sized to be configured to receive the human penis 202 (of FIG. 4). The opening 107 may have a downward angle portion that points away from the retainment mechanism 110 (of FIG. 9). The opening 107 may be shaped as a slat with a thin opening such that the human penis 202 (of FIG. 4) can be received through the opening 107 without moving significantly. In other examples, the opening 107 may have a door configuration such that the human penis 202 (of FIG. 4) may be received therethrough. In some examples, the opening 107 may include a slat or door configuration affixed to a concave portion such that when the human penis 202 (of FIG. 4) is received through the opening 107, the human penis 202 (of FIG. 4) may rest on the concave portion. It should be appreciated that the slat or door configuration of the opening 107 is contemplated, as it can accommodate any sized/shaped human penis.

In some examples, a material may be located on the interior of the opening 107 and may be used to adhere to flesh of the human penis 202 (of FIG. 4). In some examples, the material may be a soft rubber material, an elastic material, a nylon material, and/or a spandex material, among others not explicitly listed herein. In some examples, the elastic material may be an elastic spanx material. In examples, the material may be located on a portion of the interior of the opening 107. In some examples, the material may be located on an entirety of the interior of the opening 107. As depicted in FIG. 10, a rear of the third embodiment of device 100 may cover an entirety of the back side of the patient 200 (of FIG. 4).

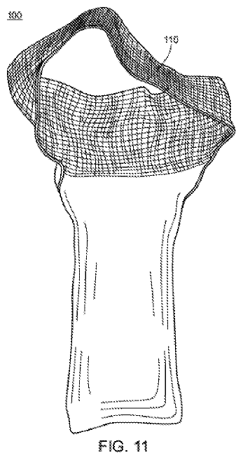

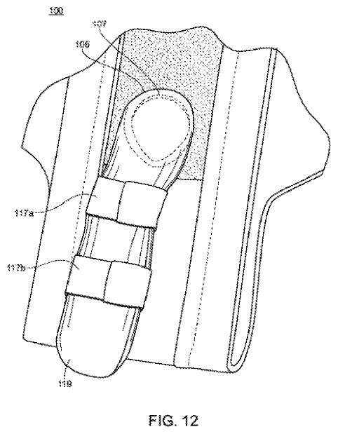

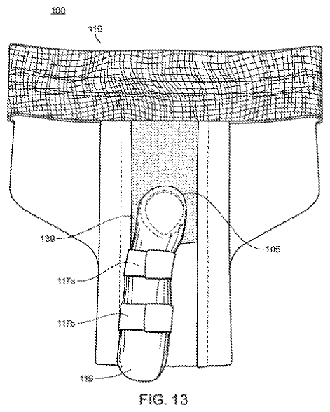

FIG. 11 depicts a perspective view of a receiving pouch of the present invention. FIG. 12 and FIG. 13 depict perspective views of a system comprising the receiving pouch of FIG. 11 and the urinary incontinence device of the third embodiment of FIG. 9 and FIG. 10 of the present invention. The device 100 of FIG. 11 and the system of FIG. 12 and FIG. 13 may be used for men who are incontinent, bedridden, or are recovering from a surgery. In some examples, the device 100 replaces a need for a catheter for an adult recovering from surgery, as the device 100 may collect urine and keep the user dry.

The device 100 (as depicted in FIG. 11) may be configured as a receiving pouch 119 and may comprise one or more materials that are approximately one inch thick. The receiving pouch 119 includes the opening 106, which is sized to receive the human penis 202 (of FIG. 4) and to collect urine from the patient 200 (of FIG. 4). One or more straps 117A, 117B (as depicted in FIG. 12 and FIG. 13) may be located on an outer surface of the receiving pouch 119 and may be configured to affix the receiving pouch 119 in a closed position once the human penis 202 (of FIG. 4) is received therein, allowing the patient 200 (of FIG. 4) to quickly and easily secure the receiving pouch 119 in a closed position.

To form the system of FIG. 12 and FIG. 13, the receiving pouch 119 of FIG. 11 may be affixed to an outer surface of a front side of the device 100 (as depicted in FIG. 9) such that the opening 107 of the device 100 of FIG. 9 affixes to the opening 106 of the receiving pouch 119 of FIG. 11, allowing the human penis 202 (of FIG. 4) to be received through the opening 107 of the device 100 of FIG. 9 and rest in the receiving pouch 119 of FIG. 11. In some examples, the receiving pouch 119 of FIG. 11 may be affixed to the front side of the device 100 (of FIG. 9) via one or more affixation means, such as an adhesive material.

The embodiments of the present invention described herein provide for a male incontinence device that may be worn by a user as needed, such as, before, during, or after surgery or by those generally having incontinence issues. Such a device is intended to replace traditional catheters which can be painful to use and can cause serious infections amongst other problems.

What is claimed is:

1. A urinary incontinence device comprising:
a pouch having at least one inner layer and an outer layer defining an opening, wherein one of the at least one inner layer is comprised of an absorbent material, and wherein the outer layer is made of a liquid impervious material;
at least one securement mechanism coupled to the pouch; and
at least one retainment mechanism configured to be coupled to the pouch.

2. The device of claim 1,
wherein a size of the opening is adjustable, and
wherein the opening is configured to receive a human penis.

3. The device of claim 1, wherein the securement mechanism comprises a first end and a second end with each of the first end and the second end coupled to the pouch thereby forming a loop.

4. The device of claim 1, further comprising:
an adhesive disposed on an outer surface of the pouch.

5. The device of claim 1, wherein the absorbent material contains sodium polyacrylate.

6. The device of claim 1, wherein there are two securement mechanisms, and wherein the at least one retainment mechanism is coupled to each of the two securement mechanisms.

7. The device of claim 2, further comprising:
flaps disposed in proximity to the opening and being configured to adjust the size of the opening.

8. A male urinary incontinence device comprising:
a pouch having an inner surface and an outer surface defining an opening with the opening being configured to receive a human penis;
an absorbent material disposed between the inner surface and the outer surface of the pouch, wherein the outer surface is made of a liquid impervious material;
two securement mechanisms coupled to the pouch, wherein each of the two securement mechanisms comprises a first end and a second end with each of the first end and the second end coupled to the pouch thereby forming a loop; and
a length of material having a first end and a second end with the first end being configured to be coupled to one of the two securement mechanisms and the second end being configured to be coupled to another of the two securement mechanisms.

9. The device of claim 8, wherein the length of material has a fastening mechanism disposed on each of the first end and the second end of the length of material.

10. The device of claim 8, wherein the outer surface comprises an outer layer and an inner layer, wherein the outer layer comprises the liquid impervious material, and wherein the inner layer comprises a cloth layer.

11. The device of claim 8, wherein the device is configured to hold up to 1 L of fluid.

12. A male urinary incontinence device comprising:
a pouch having at least one inner layer and an outer layer defining an opening with the opening being configured to receive a human penis, wherein the at least one inner layer is comprised of sodium polyacrylate, and wherein the outer layer is made of a petroleum-based material;
at least one flap disposed in proximity to the opening and being configured to adjust the size of the opening, wherein a positioning mechanism is disposed on one end of the at least one flap to secure a position of the at least one flap;
two securement mechanisms coupled to the pouch, wherein each of the two securement mechanisms comprises a first end and a second end with each of the first end and the second end coupled to the pouch thereby forming a loop;
an adhesive material being disposed on an outer layer of the pouch; and
a length of material having a first end and a second end with the first end being configured to couple to one of the two securement mechanisms and the second end being configured to couple to another of the two securement mechanisms, wherein the length of material has a fastening mechanism disposed on each of the first end and the second end of the length of material.

13. The device of claim 12, wherein the adhesive material is disposed on a back surface of the pouch.

14. The device of claim 12, wherein the inner layer is removable.

15. The device of claim 12, wherein the outer layer of the pouch is about 3 inches thick.

16. The device of claim 12, wherein a base of the pouch is wider than the opening of the pouch.

17. The device of claim 12, wherein the opening comprises a slat configuration or a door configuration affixed to a concave portion.

18. The device of claim 17, wherein the human penis rests on the concave portion once the human penis is received through the opening.

19. The device of claim 12, further comprising:
a receiving pouch affixed to the opening on an outer surface of a front side of the device, wherein the receiving pouch is configured to receive and house the human penis once received through the opening.

20. The device of claim 19, wherein the receiving pouch comprises one or more urine-absorbent materials.
